(19) United States
(12) Patent Application Publication
(10) Pub. No.: US 2020/0345590 A1
(43) Pub. Date: Nov. 5, 2020

(54) CLEANSING AND CONDITIONING TABLETS, COMPOSITIONS, AND METHODS OF MANUFACTURE THEREOF

(71) Applicant: EARTHSUDS INC., Waterloo (CA)

(72) Inventors: Marissa Vettoretti, Maples (CA); Emma Jodoin, Toronto (CA); Kayla Anareta, Cambridge (CA)

(21) Appl. No.: 16/864,734

(22) Filed: May 1, 2020

Related U.S. Application Data
(60) Provisional application No. 62/842,868, filed on May 3, 2019.

(51) Int. Cl. A61K 8/02; A61K 8/46; A61K 8/92; A61K 8/73; A61K 8/362; A61K 8/34; A61K 8/49; A61K 8/96; A61K 8/67; A61Q 5/02; A61Q 5/12; A61Q 19/10

(52) U.S. Cl. CPC A61K 8/0216; A61K 2800/524; A61K 8/922; A61K 8/732; A61K 8/362; A61K 8/342; A61K 8/345; A61K 8/498; A61K 8/965; A61K 8/678; A61Q 5/02; A61Q 5/12; A61Q 19/10; A61K 2800/43; A61K 2800/59; A61K 2800/805; A61K 8/463

(57) ABSTRACT

Cleansing and conditioning tablets and compositions are provided. The tablets are solid at room temperature and dissolve in water in one use (under about 15 seconds) to provide cleanser (e.g., shampoo or body wash) and conditioner for the hair and body. The tablet compositions are generally formed of between about 15 percent by weight to about 35 percent by weight of liquid components (i.e., in liquid state at room temperature) to allow the ingredients to be easily mixed together and allowed to set to form solid tablets without requiring compression to compact the solid ingredients together.

### FIELD

The specification relates generally to cleansing and conditioning compositions, and more particularly to cleansing and conditioning compositions for single-use tablets.

### BACKGROUND

Soaps, shampoos, and conditioners for cleansing and conditioning are often water-based compositions and come in a liquid form, requiring plastic packaging for distribution. Further, the packaging often still contains residual soap and therefore may not be recycled. The plastic packaging may end up in landfills and waterways where it breaks down into microplastics. These issues and ecological consequences are further exacerbated for single-use or travel-sized portions.

### SUMMARY

According to an aspect of the present specification, a cleansing tablet composition is provided. The cleansing tablet composition includes: at least one cleansing agent in an amount from about 45 wt. % to about 80 wt. %, the at least one cleansing agent including a surfactant; at least one moisturizing agent in an amount from about 20 wt. % to about 30 wt. %; and at least one thickening agent in an amount of up to about 30 wt. %, the at least one thickening agent to solidify the composition; and wherein between about 15% and about 35% of the composition comprises liquid components at room temperature.

According to another aspect of the present specification, another cleansing tablet composition is provided. The cleansing tablet composition includes: at least one cleansing agent in amount from about 60 wt. % to about 83 wt. %, the at least one cleansing agent including a surfactant; at least one moisturizing agent in an amount from about 17 wt. % to about 27 wt. %; and at least one thickening agent in amount of up to about 15 wt. %, the at least one thickening agent to solidify the composition; and wherein between about 15 wt. % and about 35 wt. % of the composition is in a liquid state at room temperature.

According to another aspect of the present specification, a conditioning tablet composition is provided. The conditioning tablet composition includes: at least one emulsifier in an amount from about 15 wt. % to about 35 wt. %; at least one moisturizing agent in an amount from about 30 wt. % to about 50 wt. %; and at least one thickening agent in an amount from about 20 wt. % to about 50 wt. %, the at least one thickening agent to solidify the composition; and wherein between about 15 wt. % and about 35 wt. % of the composition comprises liquid components at room temperature, and between about 20 wt. % and about 50 wt. % of the composition comprises meltable solid components, the meltable solid components are in a solid state at room temperature and have a melting point of between about 25° C. and about 75° C.

### BRIEF DESCRIPTION OF DRAWINGS

Implementations are described with reference to the following figures, in which:

FIG. 1 depicts an example flowchart of a method of manufacturing a cleansing tablet; and

FIG. 2 depicts an example flowchart of a method of manufacturing a conditioning tablet.

### DETAILED DESCRIPTION

Present solutions for decreasing the carbon and ecological footprint of shampoos, conditioners and soaps revolve around moving to biodegradable materials, as well as improving recycling mechanisms and practices. In view of the prevalence of plastic packaging in the cosmetics, including for packaging soaps, conditioners and shampoos, such solutions are often not viable or sustainable.

According to the present specification, two cleansing tablet compositions and a conditioning tablet composition are provided. In particular, the tablets compositions form tablets which are solid at room temperature and dissolve in water under pressure and agitation by a subject. More particularly, the tablets may be sized to be single-use tablets so that a single tablet may dissolve in one use (e.g., in under about 15 seconds) while providing adequate cleanser or conditioner.

To achieve this, the tablet compositions are generally formed of between about 15 percent by weight to about 35 percent by weight of liquid components. This ratio of liquid to solid components allows the tablet to be manufactured in a solid form without requiring compression to compact the powdered solid ingredients together to solidify the tablet. Further, the lack of compression allows the tablets to be dissolved in warm water under pressure and agitation applicable by a human subject without undue time and energy.

Generally, the cleansing tablet compositions include at least one cleansing agent to cleanse the hair or body of a subject, at least one moisturizing agent to increase moisture content of the hair or body of the subject, and at least one thickening agent to solidify the tablet.

The conditioning tablet composition includes at least one emulsifier to promote emulsification during manufacture, at least one moisturizing agent to increase moisture content of the hair or body of the subject, and at least one thickening agent to solidify the tablet.

Such compositions result in tablets for cleansing and conditioning the body and the hair and are convenient for consumers. Further, their solid form factor allows them to be packaged in recyclable materials and reusable cases.

As used herein, the term “about” means a variation in a numerical quantity that may occur for any reason whatsoever in the real world, such as, for example, through typical measuring and liquid/solid handling procedures used for making the compositions, through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. For example, the term “about” may mean a variation of up to ±5%.

The term “wt. %,” “weight percent,” “% w/w,” “percent by weight,” “% by weight,” and variations thereof refer to the concentration of a substance as the weight of that substance divided by the total weight of the composition and multiplied by 100.

The term “effective amount” of a compound or component means that amount which would provide the desired effect of the compound based on the compounds function and properties as would be apparent to a person of skill in the art reading the present disclosure.

As used herein, the terms “liquid components” and “solid components” refer to the physical state of a given ingredient at room temperature (i.e., between about 20° C. and about 25° C.). The solid components may further be referred to as “meltable solids” which may be melted to a liquid at temperatures between about 25° C. and about 75° C., and “powdered solids” which have melting points higher than 75° C. and hence are generally not melted during the processes described herein.

The subject components of the cleansing and conditioning tablet compositions will now be described in greater detail.

The cleansing agents are generally to cleanse the hair or body of a subject and are included in the cleansing tablet compositions. In particular, the cleansing agents may include surfactants, such as anionic surfactants, cationic surfactants, nonionic surfactants, or amphoteric surfactants. Preferably, anionic surfactants may be utilized to provide suitable cleansing properties for cleansing the hair and the body of a human subject. Preferably, the cleansing agents are also non-aqueous or substantially non-aqueous to promote the solidification of the resulting cleansing tablet and to reduce the likelihood of the cleansing tablet disintegrating and dissolving during manufacture or packaging.

Example cleansing agents include, but are not limited to, sodium lauryl sulfoacetate, sodium cocoyl isethionate, sodium laureth sulfate, sodium lauryl sulfate, sodium cocoyl glutamate, sodium lauryl sarcosinate, sodium myreth sulfate, sodium pareth sulfate, sodium stearte, sodium lauryl sulfate, coco betaine, cocamidopropylamine oxide and other suitable surfactants. Other suitable cleansing agents will also be apparent to those of skill in the art. Further, in some examples, combinations of the above-mentioned cleansing agents and/or other suitable cleansing agents may be utilized.

The cleansing agents or combinations thereof may be selected, for example, based on the desired pH, hardness or softness of the cleansing tablet composition, its ability of the cleansing tablet to dissolve in water, or other relevant characteristics of the tablet composition or the resulting tablet. The cleansing agents or combinations thereof may further be selected for chemical compatibility with the other ingredients of the cleansing and conditioning tablet.

The cleansing agents generally comprise powders and are solid components. In other examples, other cleansing agents which are liquid components may occur to those of skill in the art.

The moisturizing agents are generally to provide moisture to the hair or body of the subject and may be included in the cleansing or conditioning tablet compositions. In particular, the moisturizing agents may include blends of fatty acids, including, oleic, auric, mpristic, palmitic, stearic, rininoleic, linoleic, and linolenic fatty acids. The moisturizing agents may further assist in binding the resulting tablets together. Preferably, the moisturizing agents are also non-aqueous or substantially non-aqueous to promote the solidification of the resulting cleansing or conditioning tablet and to reduce the likelihood of the tablet disintegrating and dissolving during manufacture or packaging.

Example moisturizing agents may include, but are not limited to, avocado oil, apricot oil, argan oil, coco caprylate, marshmallow root, glycerin, grapeseed oil, dimethicone, ethylhexyl olivate, cetrimonium chloride, hyaluronic acid, various carrier oils (e.g., coconut oil, palm oil, macadamia oil, rosehip oil, hemp oil), vitamin E, aloe vera juice, butters (e.g., shea butter, mango butter, avocado butter, almond butter, soy butter, almond butter), PEG 40, silk peptide, and other suitable moisturizing agents. Other suitable moisturizing agents will also be apparent to those of skill in the art. Further, in some examples, combinations of the above-mentioned moisturizing agents and/or other suitable moisturizing agents may be utilized.

The moisturizing agents or combinations thereof may be selected, for example, based on the desired moisturizing properties of the subject (e.g., based on the purpose of the tablet to moisturize the hair or the body), and other relevant characteristics of the tablet composition or the resulting tablet. The moisturizing agents or combinations thereof may further be selected for chemical compatibility with the other ingredients.

The moisturizing agents may comprise both solid components and liquid components. Some of the solid components, such as various butters, may be solid at room temperature, but may be melted between about 25° C. and about 75° C. (also referred to herein as meltable solids) during the manufacturing process.

The emulsifiers are generally to promote oil-in-water emulsification of the oil ingredients during the manufacturing process. Preferably, the emulsifiers are also non-aqueous or substantially non-aqueous to promote the solidification of the resulting conditioning tablet and to reduce the likelihood of the conditioning tablet disintegrating and dissolving during manufacture or packaging. Example emulsifiers may include, but are not limited to, cetyl alcohol, stearyl alcohol, stearic acid, and glyceryl stearate citrate. Other suitable emulsifiers will also be apparent to those of skill in the art. Further, in some examples, combinations of the above-mentioned emulsifiers and/or other suitable emulsifiers may be utilized.

The emulsifiers or combinations thereof may be selected based on chemical compatibility with the other ingredients of the conditioning tablet, to suitably promote emulsification. The emulsifiers may generally comprise solid components which have a melting point of between about 25° C. and about 75° C.

The thickening agents are generally to thicken and solidify the tablet compositions. Example thickening agents may include, but are not limited to, tapioca starch, rice starch, cornstarch, potato starch, polyethylene glycol, guar gum, arrowroot, pectin, xanthum gum, hydroxypropyl methylcellulose, stearyl palmitate, sorbitol, sodium stearate, and other suitable thickening agents, as will be apparent to those of skill in the art. Further, in some examples, combinations of the above-mentioned thickening agents and/or other suitable thickening agents may be utilized.

The thickening agents or combinations thereof may be selected based on chemical compatibility with the other ingredients of the cleansing or conditioning tablets. In particular, the thickening agents are generally chemically inert to allow the thickening agents to be used as a filler material in the tablet compositions.

The cleansing and conditioning tablet compositions may also optionally include preservative agents and aesthetic agents.

The preservative agents may assist in preserving the cleansing and conditioning tablet compositions in their respective tablet forms to extend the shelf-life of the tablets. Example preservative agents may include, but are not limited to citric acid, gluconolactone, sodium glutamate, benzylalcohol, paraben-DU, phenoxyethanol SA, sorbic acid, propylparaben, butylparaben, isopropylparaben, methylparaben, and ethylparaben. Other suitable preservative agents will also be apparent to those of skill in the art. Further, in some examples, combinations of the above-mentioned preservative agents and/or other suitable preservative agents may be utilized.

The preservative agents or combinations thereof may be selected based on chemical compatibility with the other ingredients of the cleansing or conditioning tablets. In particular, the preservative agents may be selected to preserve the active ingredients (i.e., the cleansing agents, moisturizing agents, thickening agents, and emulsifiers) while being substantially inert with respect to the active ingredients so as to maintain their functionality.

The aesthetic agents may provide aesthetic qualities to the resulting cleansing and conditioning tablets, such as providing fragrances, coloring or the like. For example, the aesthetic agents may include carrier oils, essential oils, absolute oils, or fragrance oils to provide fragrance to the tablets. The aesthetic agents may further include coloring agents, such as colored mica powder, or other suitable coloring agents, as will be apparent to those of skill in the art. In some examples, combinations of aesthetic agents, including different types of aesthetic agents or multiple of the same type of aesthetic agent may be utilized, for example to create blended fragrances or specific colors.

The aesthetic agents or combinations thereof may be selected based on the desired aesthetics of the resulting tablet, as well as chemical compatibility with the other ingredients of the cleansing or conditioning tablets. In particular, the aesthetic agents may be selected to be inert with respect to the active ingredients (i.e., the cleansing agents, moisturizing agents, thickening agents, and emulsifiers) so as to maintain their functionality.

Having described the components of the cleansing and conditioning tablet compositions, the compositions, the tablets, and the method of manufacturing the tablets will now be described in greater detail.

According to a first example, a first cleansing tablet composition is provided which is to form a first cleansing tablet for shampooing the hair of a subject. The first cleansing tablet composition includes at least one cleansing agent in an amount from about 45 wt. % to about 80 wt. %, at least one moisturizing agent in an amount from about 20 wt. % to about 30 wt. % and at least one thickening agent in an amount of up to about 30 wt. %. Further, the composition includes between about 15 wt. % and about 35 wt. % of liquid components (i.e., components in liquid state at room temperature—between about 20° C. and about 25° C.), with the remainder of the ingredients being solid components.

The at least one cleansing agent is provided in an effective amount to provide cleansing properties to the cleansing tablet composition. In some examples, combinations of cleansing agents may be utilized to promote the dissolution of the solid component cleansing agent (often provided in powdered forms) in the liquid components of the cleansing tablet compositions. For example, two different surfactants, having for example, different densities, chemical compositions, polarity, or the like, may be selected to work together to both cleanse the subject and dissolve in the liquid components of the composition. Preferably, anionic surfactants, and in particular, surfactants which are milder or gentler on human hair or skin are used as the cleansing agents to avoid drying and dehydrating the hair or scalp of the subject.

The at least one moisturizing agent is provided in an effective amount to moisturize the hair during use of the cleansing tablet. In some examples, combinations of moisturizing agents may be utilized based on the different effects of the moisturizing agent on the hair of a subject. For example, different carrier oils and combinations thereof may be selected based on their promotion of hair strength, hair growth, hair moisture, hair softness, or other beneficial properties, as will be apparent to those of skill in the art. The at least one moisturizing agent, which may include various carrier oils, butters, and the like, often provides the majority of the liquid components of the cleansing tablet composition and serves to bind the cleansing tablet composition during manufacture.

The thickening agent may be provided in an effective amount to thicken and solidify the cleansing tablet composition. Specifically, when its components are mixed together, the cleansing tablet composition forms an intermediate paste, which has a soft, dough-like, malleable consistency under pressure and retains its shape when left at rest. The thickening agent therefore promotes the thickening of the composition to form the intermediate paste, as well as the eventual solidification of the intermediate paste into a solid tablet. In some examples, rather than specifically providing a thickening agent, additional surfactant (i.e., beyond the effective amount required to provide cleansing properties to the cleansing tablet composition), may be added to increase the powdered solid components of the composition to obtain a composition having between about 15 wt. % and about 35 wt. % of liquid components, with the remainder of the components being solid components. That is, additional surfactant may effectively act as a thickener to promote the thickening of the composition to form the intermediate paste.

Optionally, the composition may further include at least one preservative agent in an amount from about 0.01 wt. % to about 4 wt. %, and/or at least one aesthetic agent in an amount from about 0.001 wt. % to about 4 wt. %.

Preferably, the at least one cleansing agent is provided in an amount from about 45 wt. % to about 55 wt. %, the at least one moisturizing agent is provided in an amount from about 21 wt. % to about 29 wt. %, the at least one thickening agent is provided in an amount from about 17 wt. % to about 27 wt. %, the at least one preservative agent is provided in an amount from about 0.01 wt. % to about 4 wt. %, and the at least one aesthetic agent is provided in an amount from about 0.01 wt. % and about 4 wt. %. Preferably, the at least one cleansing agent includes sodium lauryl sulfoacetate and sodium cocoyl isethionate; the at least one moisturizing agent includes avocado oil, apricot oil, argan oil, coco caprylate, marshmallow root and glycerin; the at least one thickening agent includes tapioca starch; the at least one preservative agent includes citric acid and gluconolactone; and the at least one aesthetic agent includes peppermint essential oil, and cedarwood essential oil.

As will be appreciated, different variants of the presently described composition may utilize different component ingredients (e.g., different cleansing agents, moisturizing agents, aesthetic agents, etc.) to target specific hair conditions or desired aesthetics. For example, different moisturizing agents may be provided to cleanse and moisturize dry hair, oily hair, curly hair, or the like. Similarly, different coloring agents may be utilized to represent the fragrances associated with the cleansing tablet, or may be omitted altogether to represent an unscented cleansing tablet.

An example cleansing tablet composition (COMPOSITION 1) forming a shampoo was prepared according to Table 1. This example is intended to be representative of a specific example of the invention and is not intended to limit the scope of the invention as described and claimed herein.

TABLE 1
Cleansing Composition 1 (Shampoo)

| | | State at room | Amount |
|---|---|---|---|
| Ingredient | Classification | temperature | (wt. %) |
| SLSA (sodium lauryl | Cleansing agent | Solid (powder) | 40.14 |
| sulfoacetate) 1 | | | |
| SLSA 2 | Cleansing agent | Solid (powder) | 8.7 |
| Sodium cocoyl | Cleansing agent | Solid (powder) | 2.74 |
| isethionate | | | |
| Marshmallow root | Moisturizing agent | Solid (powder) | 0.68 |
| Glycerin | Moisturizing agent | Liquid | 9.97 |
| Coco caprylate | Moisturizing agent | Liquid | 4.8 |
| Avocado oil | Moisturizing agent | Liquid | 4.7 |
| Apricot oil | Moisturizing agent | Liquid | 3.65 |
| Argan oil | Moisturizing agent | Liquid | 1.34 |
| Tapioca starch | Thickening agent | Solid (powder) | 21.85 |
| Citric acid | Preservative agent | Solid (powder) | 0.96 |
| Gluconolactone | Preservative agent | Solid (powder) | 0.33 |
| Cedarwood essential oil | Aesthetic agent | Liquid | 0.07 |
| Peppermint essential oil | Aesthetic agent | Liquid | 0.07 |

As can be seen, about 25 wt. % of the components of COMPOSITION 1 are liquid components, while the remainder are solid components. This ratio of liquid to solid components allows the tablet to be manufactured by simply mixing the ingredients, and allowed to set and solidify to form a cleansing tablet which is solid at room temperature (i.e., between about 20° C. and about 25° C.). Further, the tablet having the composition described in Table 1 is easily dissolved in water (e.g., preferably in warm water between about 35° C. to about 43° C.) to form a shampoo for cleansing the hair for a human subject. In use, in some examples, the human subject may apply pressure and agitation to the cleansing tablet to assist in dissolving the cleansing tablet.

The cleansing tablets are further sized to be single-use tablets so that a single tablet may dissolve in one use to provide adequate shampoo for a typical human subject. Specifically, the tablets may be approximately cuboid in shape and have dimensions of about 1.5 cm by about 1.5 cm by about 1 cm. Tablets of this size may thus dissolve in under about 15 seconds to be used as shampoo for shampooing the hair of the human subject.

According to a second example, a second cleansing tablet composition is provided to form a first cleansing tablet for cleansing the body of a subject (i.e., a body wash). The second cleansing tablet composition includes at least one cleansing agent in amount from about 60 wt. % to about 83 wt. %; at least one moisturizing agent in an amount from about 17 wt. % to about 27 wt. %; and at least one thickening agent in amount of up to about 15 wt. %, the at least one thickening agent to solidify the composition. Further, the composition includes between about 15 wt. % and about 35 wt. % liquid components (i.e. components in liquid state at room temperature), with the remainder of the ingredients being solid components.

The at least one cleansing agent is provided in an effective amount to provide cleansing properties to the cleansing tablet composition. In some examples, combinations of cleansing agents may be utilized to promote the dissolution of the solid component cleansing agent (often provided in powdered forms) in the liquid components of the cleansing tablet compositions. For example, two different surfactants, having for example, different densities, chemical compositions, polarity, or the like, may be selected to work together to both cleanse the subject and dissolve in the liquid components of the composition. Preferably, anionic surfactants, and in particular, surfactants which are milder or gentler on human skin are used as the cleansing agents to avoid drying and dehydrating the skin of the subject.

The at least one moisturizing agent is provided in an effective amount to moisturize the skin during use of the cleansing tablet. In some examples, combinations of moisturizing agents may be utilized based on the different effects of the moisturizing agent on the skin of a subject. For example, different carrier oils and combinations thereof may be selected based on their promotion of skin moisture, skin softness, treatment of skin conditions, or other beneficial properties, as will be apparent to those of skill in the art. The at least one moisturizing agent, which may include various carrier oils, butters, and the like, often provides the majority of the liquid components of the cleansing tablet composition and serves to bind the cleansing tablet composition during manufacture.

The thickening agent may be provided in an effective amount to thicken and solidify the cleansing tablet composition. Specifically, when its components are mixed together, the cleansing tablet composition forms an intermediate paste, which has a soft, dough-like, malleable consistency under pressure and retains its shape when left at rest. The thickening agent therefore promotes the thickening of the composition to form the intermediate paste, as well as the eventual solidification of the intermediate paste into a solid tablet. In some examples, rather than specifically providing a thickening agent, additional surfactant (i.e., beyond the effective amount required to provide cleansing properties to the cleansing tablet composition), may be added to increase the powdered solid components of the composition to obtain a composition having between about 15 wt. % and about 35 wt. % of liquid components, with the remainder of the components being solid components. That is, additional surfactant may effectively act as a thickener to promote the thickening of the composition to form the intermediate paste.

Optionally, the composition may further include at least one preservative agent in an amount from about 0.01 wt. % to about 4 wt. %, and/or at least one aesthetic agent in an amount from about 0.001 wt. % to about 4 wt. %.

Preferably, the at least one cleansing agent is provided in an amount from about 63 wt. % to about 73 wt. %; the at least one moisturizing agent is provided in an amount from about 18 wt. % to about 26 wt. %; the at least one thickening agent is provided in an amount from about 4 wt. % to about 14 wt. %; the at least one preservative agent is provided in an amount from about 0.01 wt. % to about 4 wt. %; and the at least one aesthetic agent is provided in an amount from about 0.01 wt. % to about 4 wt. %. Preferably, the at least one cleansing agent includes SLSA and sodium cocoyl glutamate; the at least one moisturizing agent includes avocado oil, jajoba oil, grapeseed oil, burdock root, green tea extract, coco caprylate, and glycerin; the at least one thickening agent includes tapioca starch; the at least one preservative agent includes citric acid and gluconolactone; and the at least one aesthetic agent includes peppermint essential oil, cedarwood essential oil, and blue mica powder.

As can be seen, the components of the second cleansing tablet composition are similar to those of the first cleansing tablet composition. The specific selection of components may differ according the different properties and effects of the components on human hair versus human skin. Further, as with the shampoo, different variants of the presently described composition may utilize different component ingredients to target specific skin conditions or desired aesthetics. For example, different moisturizing agents may be provided to target dry skin, various skin conditions, or the like. Similarly, different coloring agents may be utilized to represent the fragrances associated with the cleansing tablet, or may be omitted altogether to represent an unscented cleansing tablet.

An example cleansing tablet composition (COMPOSITION 2) forming a body wash was prepared according to Table 2. This example is intended to be representative of a specific example of the invention and is not intended to limit the scope of the invention as described and claimed herein.

TABLE 2
Cleansing Composition 2 (Body wash)

| | | State at room | Amount |
|---|---|---|---|
| Ingredient | Classification | temperature | (wt. %) |
| SLSA (sodium lauryl | Cleansing agent | Solid (powder) | 44.9 |
| sulfoacetate) 1 | | | |
| SLSA 2 | Cleansing agent | Solid (powder) | 21.25 |
| Sodium cocoyl glutamate | Cleansing agent | Liquid | 1.15 |
| Glycerin | Moisturizing agent | Liquid | 8.37 |
| Coco caprylate | Moisturizing agent | Liquid | 0.48 |
| Avocado oil | Moisturizing agent | Liquid | 9.84 |
| Jajoba oil | Moisturizing agent | Liquid | 1.73 |
| Grapeseed oil | Moisturizing agent | Liquid | 1.73 |
| Burdock root | Moisturizing agent | Solid (powder) | 0.27 |
| Green tea extract | Moisturizing agent | Solid (powder) | 0.27 |
| Tapioca starch | Thickening agent | Solid (powder) | 8.36 |
| Citric acid | Preservative agent | Solid (powder) | 0.48 |
| Gluconolactone | Preservative agent | Solid (powder) | 0.96 |
| Cedarwood essential oil | Aesthetic agent | Liquid | 0.07 |
| Peppermint essential oil | Aesthetic agent | Liquid | 0.07 |
| Blue mica powder | Aesthetic agent | Solid (powder) | 0.06 |

As can be seen, about 23 wt. % of the components of COMPOSITION 2 are liquid components, while the remainder are solid components. This ratio of liquid to solid components allows the tablet to be manufactured by simply mixing the ingredients, and allowed to set and solidify to form a cleansing tablet which is solid at room temperature (i.e., between about 20° C. and about 25° C.). Further, the tablet having the composition described in Table 2 is easily dissolved in water (e.g., preferably in warm water between about 35° C. to about 43° C.) to form a body wash for cleansing the body of a human subject. In use, in some examples, the human subject may apply pressure and agitation to the cleansing tablet to assist in dissolving the cleansing tablet.

The body wash cleansing tablets, similarly to the shampoo cleansing tablets, are further sized to be single-use tablets so that a single tablet may dissolve in one use to provide adequate body wash for a typical human subject. Specifically, the tablets may be approximately cuboid in shape and have dimensions of about 1.5 cm by about 1.5 cm by about 1 cm. Tablets of this size may dissolve in under about 15 seconds to form a body wash for cleansing the body of the human subject.

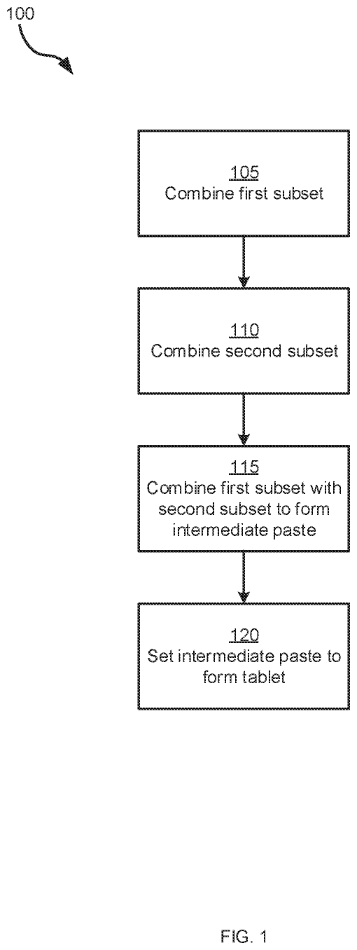

Referring to FIG. 1, a flowchart depicting an example method 100 of manufacturing a cleansing tablet (e.g. the shampoo cleansing tablet or the body wash cleansing tablet described above) is depicted. At block 105, a first subset of the composition components (i.e., the at least one cleansing agent, the at least one moisturizing agent, the at least one thickening agent, and any included preservative agents or aesthetic agents) are combined. The first subset includes solid components and only solid components.

At block 110, a second subset of the remaining composition components are combined. The second subset includes primarily liquid components and may include a portion of the solid components. More particularly, the second subset, when combined, remains a liquid mixture at room temperature.

At block 115, the first subset and the second subset are combined to form an intermediate paste. Specifically, the intermediate paste has a soft, dough-like, malleable consistency, and may be readily deformed under hand pressure or manipulation applicable by a human operator yet retains its shape when left at rest.

At block 120, the intermediate paste is set for at least about 2 hours to form a solid form. Specifically, the solid form comprises the resulting cleansing tablet. The intermediate paste may be set, for example, in molds shaped and sized as desired for the cleansing tablet. In other examples, the intermediate paste may be extruded from an extruder, cut to the desired size and left to set. Further, in some examples, the intermediate paste may be refrigerated to expedite setting. However, the cleansing tablet, once set, is a solid at room temperature. Other manners of setting the intermediate paste to produce the cleansing tablet will also be apparent to those of skill in the art.

According to a third example, a conditioning tablet composition is provided to form a conditioning tablet for conditioning the hair of a subject. The conditioning composition includes at least one emulsifier in an amount from about 15 wt. % to about 35 wt. %; at least one moisturizing agent in an amount from about 30 wt. % to about 50 wt. %; and at least one thickening agent in an amount from about 20 wt. % to about 50 wt. %. Further, the composition includes between about 15 wt. % and about 35 wt. % of the composition comprises liquid components at room temperature, and between about 20 wt. % and about 50 wt. % of the composition comprises solid components at room temperature having a melting point of between about 25° C. and about 75° C.

The at least one emulsifier is provided in an effective amount to promote emulsification of the ingredients during the manufacturing process. Specifically, during manufacture of the conditioning tablet, the conditioning tablet composition forms an intermediate lotion, in which the insoluble solid ingredients are emulsified or suspended in the liquid components to form a lotion. The emulsifier therefore promotes the emulsification of said solid ingredients in the liquid ingredients. Specifically, the emulsifier may be an oil-in-water emulsifier to promote the emulsification of the oil ingredients in the liquid ingredients. In some examples, combinations of emulsifiers may be utilized.

The at least one moisturizing agent is provided in an effective amount to moisturize the hair during use of the conditioning tablet. In some examples, combinations of moisturizing agents may be utilized based on the different effects of the moisturizing agent on the hair of a subject. For example, different carrier oils and combinations thereof may be selected based on their promotion of hair strength, hair growth, hair moisture, hair softness, or other beneficial properties, as will be apparent to those of skill in the art. The at least one moisturizing agent, which may include various carrier oils, butters, and the like, often provides the majority of the liquid components of the cleansing tablet composition and serves to bind the cleansing tablet composition during manufacture.

The thickening agent may be provided in an effective amount to thicken and solidify the conditioning tablet composition. Specifically, during manufacture of the conditioning tablet, the conditioning tablet composition forms an intermediate paste, which has a soft, dough-like, malleable consistency under pressure and retains its shape when left at rest. The thickening agent therefore promotes the thickening of the composition to form the intermediate paste, as well as the eventual solidification of the intermediate paste into a solid tablet.

Optionally, the composition may further include at least one preservative agent in an amount from about 0.01 wt. % to about 4 wt. %, and/or at least one aesthetic agent in an amount from about 0.001 wt. % to about 4 wt. %.

Preferably, the at least one emulsifier is provided in an amount from about 17 wt. % to about 27 wt. %; the at least one moisturizing agent is provided in an amount from about 34 wt. % to about 44 wt. %; the at least one thickening agent is provided in an amount from about 34 wt. % to about 44 wt. %; the at least one preservative agent is provided in an amount from about 0.01 wt. % to about 4 wt. %; and the at least one aesthetic agent is provided in an amount from about 0.01 wt. % to about 4 wt. %. Preferably, the at least one emulsifier includes cetyl alcohol; the at least one moisturizing agent includes avocado oil, argan oil, apricot oil, mango butter, vitamin E, coco caprylate, and marshmallow root; the at least one thickening agent includes tapioca starch; the at least one preservative agent includes gluconolactone; and the at least one aesthetic agent includes peppermint essential oil, cedarwood essential oil, and yellow mica powder.

As will be appreciated, as with the cleansing tablet compositions, different variants of the presently described conditioning tablet composition may utilize different component ingredients (e.g., different moisturizing agents, aesthetic agents, etc.) to target specific hair conditions or desired aesthetics. For example, different moisturizing agents may be provided to condition and moisturize dry hair, oily hair, curly hair, or the like. Similarly, different coloring agents may be utilized to represent the fragrances associated with the conditioning tablet, or may be omitted altogether to represent an unscented conditioning tablet.

An example conditioning tablet composition (COMPOSITION 3) forming a conditioner was prepared according to Table 3. This example is intended to be representative of a specific example of the invention and is not intended to limit the scope of the invention as described and claimed herein.

TABLE 3
Conditioning Composition (Conditioner)

| | | State at room | Amount |
|---|---|---|---|
| Ingredient | Classification | temperature | (wt. %) |
| Cetyl alcohol | Emulsifier | Solid | 21.89 |
| | | (meltable) | |
| Avocado oil | Moisturizing agent | Liquid | 11.19 |
| Argan oil | Moisturizing agent | Liquid | 2.43 |
| Apricot oil | Moisturizing agent | Liquid | 2.43 |
| Mango butter | Moisturizing agent | Solid | 15.57 |
| | | (meltable) | |
| Vitamin E | Moisturizing agent | Liquid | 0.97 |
| Coco caprylate | Moisturizing agent | Liquid | 5.84 |
| Marshmallow root | Moisturizing agent | Solid (powder) | 0.24 |
| Tapioca starch | Thickening agent | Solid (powder) | 38.91 |
| Gluconolactone | Preservative agent | Solid (powder) | 0.19 |
| Cedarwood | Aesthetic agent | Liquid | 0.07 |
| essential oil | | | |
| Peppermint | Aesthetic agent | Liquid | 0.07 |
| essential oil | | | |
| Yellow mica powder | Aesthetic agent | Solid (powder) | 0.19 |

As can be seen, about 23 wt. % of the components of COMPOSITION 3 are liquid components, about 37 wt. % of the components of COMPOSITION 3 are meltable solid components, and the remainder are powdered solid components. This ratio of liquid to meltable solid to powdered solid components allows the tablet to be manufactured by first forming an intermediate lotion, next forming an intermediate paste, and allowing the paste to set and solidify to form a conditioning tablet which is solid at room temperature. Further, the tablet having the composition described in Table 3 is easily dissolved in water, and preferably warm water, to form a conditioner for conditioning the hair of a human subject. In use, in some examples, the human subject may apply pressure and agitation to the conditioning tablet to assist in dissolving the conditioning tablet.

The conditioning tablets are also sized to be single-use tablets so that a single tablet may dissolve in one use to provide adequate conditioner for a typical human subject. Specifically, the tablets may be approximately cuboid in shape and have dimensions of about 1.5 cm by about 1.5 cm by about 1 cm. Tablets of this size may dissolve in under about 15 seconds to form the conditioner for conditioning the hair of a human subject.

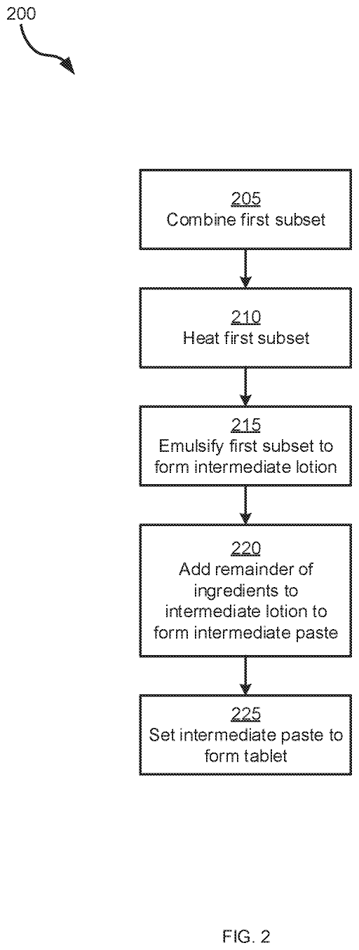

Referring now to FIG. 2, a flowchart depicting an example method 200 of manufacturing a conditioning tablet, such as the one described above, is depicted. At block 205, a first subset of the at least one emulsifier and the at least one moisturizing agent. Specifically, the first subset includes the meltable solid components and the liquid components in which the meltable solid components are to be emulsified. This may include for example, carrier oils (e.g., various moisturizing agents, essential oils, and the like).

At block 210, the first subset is heated to melt the meltable solid components.

At block 215, the first subset is whisked (or otherwise blended) as the meltable solid components cool and revert to their solid form to emulsify the meltable solid components in the liquid components of the first subset. More specifically, an intermediate lotion is formed.

At block 220, the remainder of the ingredients are added to the intermediate lotion. In some examples, the remainder of the ingredients may be separately combined into a second subset including only powdered solid components and a third subset including primarily liquid components. The third subset may include a portion of the powdered solid components, but remains a liquid mixture at room temperature when combined. Upon the combination of the remainder of the ingredients with the intermediate lotion, an intermediate paste is formed. Specifically, the intermediate paste has a soft, dough-like, malleable consistency, and may be readily deformed under hand pressure or manipulation applicable by a human operator yet retains its shape when left at rest.

At block 225, the intermediate paste is set for at least about 4 hours to form a solid form. Specifically, the solid form comprises the resulting conditioning tablet. The intermediate paste may be set, for example, in molds shaped and sized as desired for the conditioning tablet. In other examples, the intermediate paste may be extruded from an extruder, cut to the desired size and left to set. Further, in some examples, the intermediate paste may be refrigerated to expedite setting. However, the conditioning tablet, once set, is a solid at room temperature. Other manners of setting the intermediate paste to produce the conditioning tablet will also be apparent to those of skill in the art.

As described above, cleansing and conditioning tablet compositions are provided which may be used to form cleansing and conditioning tablets. The tablets are solid at room temperature for ease of manipulation and handling by human subjects and dissolve in warm water to form body wash, shampoo, and conditioner for cleansing and conditioning the hair and body of the human subject. The solid form factor of the tablets improves the efficiency and eco-friendliness of packaging, and increases convenience and versatility for the user. Further, the ratio of liquid to solid components of the composition allows for easier manufacturing processes requiring minimal machinery, as well as contributing to increased ease of use and convenience for the user to utilize the tablet (i.e., by simply dissolving in warm water).

The scope of the claims should not be limited by the embodiments set forth in the above examples but should be given the broadest interpretation consistent with the description as a whole.

1. A cleansing tablet composition comprising:
at least one cleansing agent in an amount from about 45 wt. % to about 80 wt. %, the at least one cleansing agent including a surfactant;
at least one moisturizing agent in an amount from about 20 wt. % to about 30 wt. %; and
at least one thickening agent in an amount of up to about 30 wt. %, the at least one thickening agent to solidify the composition; and
wherein between about 15% and about 35% of the composition comprises liquid components at room temperature.

2. The composition of claim 1, wherein the at least one cleansing agent is selected from the group consisting of: sodium lauryl sulfoacetate, sodium cocoyl isethionate, sodium laureth sulfate, sodium lauryl sulfate, sodium cocoyl glutamate, sodium lauryl sarcosinate, sodium myreth sulfate, sodium pareth sulfate, sodium stearate, sodium lauryl sulfate, coco betaine, and cocamidopropylamine oxide.

3. The composition of claim 1, wherein the at least one moisturizing agent is selected from the group consisting of: avocado oil, apricot oil, argan oil, coco caprylate, marshmallow root, glycerin, grapeseed oil, dimethicone, ethylhexyl olivate, cetrimonium chloride, hyaluronic acid, coconut oil, palm oil, macadamia oil, rosehip oil, hemp oil, vitamin E, aloe vera juice, shea butter, mango butter, avocado butter, almond butter, soy butter, almond butter, PEG 40, and silk peptide.

4. The composition of claim 1, wherein the at least one thickening agent is selected from the group consisting of: tapioca starch, rice starch, cornstarch, potato starch, polyethylene glycol, guar gum, arrowroot, pectin, xanthum gum, hydroxypropyl methylcellulose, stearyl palmitate, sorbitol, and sodium stearate.

5. The composition of claim 1, further comprising at least one preservative agent in an amount from about 0.01 wt. % to about 4 wt. %.

6. The composition of claim 5, wherein the at least one preservative agent is selected from the group consisting of: citric acid, gluconolactone, sodium glutamate, benzylalcohol, paraben-DU, phenoxyethanol SA, sorbic acid, propylparaben, butylparaben, isopropylparaben, methylparaben, and ethylparaben.

7. The composition of claim 1, further comprising at least one aesthetic agent in an amount from about 0.001 wt. % to about 4 wt. %.

8. The composition of claim 7, wherein the at least one aesthetic agent is selected from the group consisting of: coloring agents; essential oils, absolute oils, and fragrance oils.

9. The composition of claim 1, wherein the at least one cleansing agent, the at least one moisturizing agent and the at least one thickening agent are non-aqueous.

10. The composition of claim 1, wherein:
the at least one cleansing agent is in an amount from about 45 wt. % to about 55 wt. %;
the at least one moisturizing agent is in an amount from about 21 wt. % to about 29 wt. %;
the at least one thickening agent is in an amount from about 17 wt. % to about 27 wt. %;
and wherein the composition further comprises:
at least one preservative agent in an amount from about 0.01 wt. % to about 4 wt. %; and
at least one aesthetic agent in an amount from about 0.01 wt. % to about 4 wt. %.

11. A cleansing tablet comprising:
the cleansing tablet composition of claim 1; and
wherein the tablet comprises a solid at room temperature and dissolves in water to form a shampoo for cleansing hair.

12. The cleansing tablet of claim 11, wherein the tablet is approximately cuboid in shape, having dimensions of about 1.5 cm by about 1.5 cm by about 1 cm.

13. A method of manufacturing the cleansing tablet of claim 11, the method comprising:
combining a first subset of the at least one cleansing agent, the at least one moisturizing agent, and the at least one thickening agent, the first subset comprising solid components;
combining, separately from the first subset, a second subset comprising a remainder of the at least one cleansing agent, the at least one moisturizing agent, and the at least one thickening agent, the second subset forming a liquid mixture at room temperature;
combining the first subset with the second subset to form an intermediate paste; and
setting the intermediate paste for at least about 2 hours to a solid form to produce the cleansing tablet.

14. A cleansing tablet composition comprising:
at least one cleansing agent in amount from about 60 wt. % to about 83 wt. %, the at least one cleansing agent including a surfactant;
at least one moisturizing agent in an amount from about 17 wt. % to about 27 wt. %; and
at least one thickening agent in amount of up to about 15 wt. %, the at least one thickening agent to solidify the composition; and
wherein between about 15 wt. % and about 35 wt. % of the composition is in a liquid state at room temperature.

15. The composition of claim 14, wherein the at least one cleansing agent is selected from the group consisting of: sodium lauryl sulfoacetate, sodium cocoyl isethionate, sodium laureth sulfate, sodium lauryl sulfate, sodium cocoyl glutamate, sodium lauryl sarcosinate, sodium myreth sulfate, sodium pareth sulfate, sodium stearte, sodium lauryl sulfate, coco betaine, and cocamidopropylamine oxide.

16. The composition of claim 14, wherein the at least one moisturizing agent is selected from the group consisting of: avocado oil, apricot oil, argan oil, coco caprylate, marshmallow root, glycerin, grapeseed oil, dimethicone, ethylhexyl olivate, cetrimonium chloride, hyaluronic acid, coconut oil, palm oil, macadamia oil, rosehip oil, hemp oil, vitamin E, aloe vera juice, shea butter, mango butter, avocado butter, almond butter, soy butter, almond butter, PEG 40, and silk peptide.

17. The composition of claim 14, wherein the at least one thickening agent is selected from the group consisting of: tapioca starch, rice starch, cornstarch, potato starch, polyethylene glycol, guar gum, arrowroot, pectin, xanthum gum, hydroxypropyl methylcellulose, stearyl palmitate, sorbitol, and sodium stearate.

18. The composition of claim 14, further comprising at least one preservative agent in an amount from about 0.01 wt. % to about 4 wt. %.

19. The composition of claim 18, wherein the at least one preservative agent is selected from the group consisting of: citric acid, gluconolactone, sodium glutamate, benzylalcohol, paraben-DU, phenoxyethanol SA, sorbic acid, propylparaben, butylparaben, isopropylparaben, methylparaben, and ethylparaben.

20. The composition of claim 14, further comprising at least one aesthetic agent in an amount from about 0.001 wt. % to about 4 wt. %.

21. The composition of claim 20, wherein the at least one aesthetic agent is selected from the group consisting of: coloring agents; essential oils, and fragrance oils.

22. The composition of claim 14, wherein the at least one cleansing agent, the at least one moisturizing agent, and the at least one thickening agent are non-aqueous.

23. The composition of claim 14, wherein:
the at least one cleansing agent is in an amount from about 63 wt. % to about 73 wt. %;
the at least one moisturizing agent is in an amount from about 18 wt. % to about 26 wt. %;
the at least one thickening agent is in an amount from about 4 wt. % to about 14 wt. %;
and wherein the composition further comprises:
at least one preservative agent in an amount from about 0.01 wt. % to about 4 wt. %; and
at least one aesthetic agent in an amount from about 0.01 wt. % to about 4 wt. %.

24. A cleansing tablet comprising:
the cleansing tablet composition of claim 14; and
wherein the tablet comprises a solid at room temperature, and dissolves in water to form a body wash for cleansing a subject.

25. The cleansing tablet of claim 24, wherein the tablet is approximately cuboid in shape, having dimensions of about 1.5 cm by about 1.5 cm by about 1 cm.

26. A method of manufacturing the cleansing tablet of claim 24, the method comprising:
combining a first subset of the at least one cleansing agent, the at least one moisturizing agent, and the at least one thickening agent, the first subset comprising liquid components;
combining, separately from the first subset, a second subset of the at least one cleansing agent, the at least one moisturizing agent, and the at least one thickening agent, the second subset comprising solid components;
combining the first subset with the second subset to form an intermediate paste; and
setting the intermediate paste for at least about 2 hours to a solid form to produce the cleansing tablet.

27. A conditioning tablet composition comprising:
at least one emulsifier in an amount from about 15 wt. % to about 35 wt. %;
at least one moisturizing agent in an amount from about 30 wt. % to about 50 wt. %; and
at least one thickening agent in an amount from about 20 wt. % to about 50 wt. %, the at least one thickening agent to solidify the composition; and
wherein between about 15 wt. % and about 35 wt. % of the composition comprises liquid components at room temperature, and between about 20 wt. % and about 50 wt. % of the composition comprises meltable solid components, the meltable solid components are in a solid state at room temperature and have a melting point of between about 25° C. and about 75° C.

28. The composition of claim 27, wherein the at least one emulsifier is selected from the group consisting of: cetyl alcohol, stearyl alcohol, stearic acid, and glyceryl stearate citrate.

29. The composition of claim 27, wherein the at least one moisturizing agent is selected from the group consisting of: avocado oil, apricot oil, argan oil, coco caprylate, marshmallow root, glycerin, grapeseed oil, dimethicone, ethylhexyl olivate, cetrimonium chloride, hyaluronic acid, coconut oil, palm oil, macadamia oil, rosehip oil, hemp oil, vitamin E, aloe vera juice, shea butter, mango butter, avocado butter, almond butter, soy butter, almond butter, PEG 40, and silk peptide.

30. The composition of claim 27, wherein the at least one thickening agent is selected from the group consisting of: tapioca starch, rice starch, cornstarch, potato starch, polyethylene glycol, guar gum, arrowroot, pectin, xanthum gum, hydroxypropyl methylcellulose, stearyl palmitate, sorbitol, and sodium stearate.

31. The composition of claim 27, further comprising at least one preservative agent in an amount from about 0.01 wt. % to about 4 wt. %.

32. The composition of claim 31, wherein the at least one preservative agent is selected from the group consisting of: citric acid, gluconolactone, sodium glutamate, benzylalcohol, paraben-DU, phenoxyethanol SA, sorbic acid, propylparaben, butylparaben, isopropylparaben, methylparaben, and ethylparaben.

33. The composition of claim 27, further comprising at least one aesthetic agent in an amount from about 0.001 wt. % to about 4 wt. %.

34. The composition of claim 33, wherein the at least one aesthetic agent is selected from the group consisting of: coloring agents; essential oils, and fragrance oils.

35. The composition of claim 27, wherein the at least one emulsifier, the at least one moisturizing agent, and the at least one thickening agent are non-aqueous.

36. The composition of claim 27, wherein:
the at least one emulsifier is in an amount from about 17 wt. % to about 27 wt. %;
the at least one moisturizing agent is in an amount from about 34 wt. % to about 44 wt. %;
the at least one thickening agent is in an amount from about 34 wt. % to about 44 wt. %; and
wherein the composition further comprises:
at least one preservative agent in an amount from about 0.01 wt. % to about 4 wt. %; and
at least one aesthetic agent in an amount from about 0.01 wt. % to about 4 wt. %.

37. A conditioning tablet comprising:
the conditioning tablet composition of claim 27; and
wherein the tablet comprises a solid at room temperature, and dissolves in water to form a conditioner for hair.

38. The conditioning tablet of claim 37, wherein the tablet is approximately cuboid in shape, having dimensions of about 1.5 cm by about 1.5 cm by about 1 cm.

39. A method of manufacturing the conditioning tablet of claim 37, the method comprising:
combining a first subset of the at least one emulsifier and the at least one moisturizing agent, the first subset comprising meltable solid components and liquid components;
heating the first subset to melt the meltable solid components of the first subset;
whisking the melted meltable solid components to emulsify the meltable solid components of the first subset with the liquid components of the first subset to form an intermediate lotion;
adding a remainder of the at least one emulsifier, the at least one moisturizing agent, and the at least one thickening agent to the intermediate lotion to form an intermediate paste;
setting the intermediate paste for at least about 4 hours to a solid form to produce the conditioning tablet.
